(19) United States
(12) Patent Application Publication
(10) Pub. No.: US 2026/0294928 A1
(43) Pub. Date: Oct. 1, 2026

(54) BTK INHIBITOR FOR TREATING MULTIPLE SCLEROSIS

(71) Applicant: BIOGEN MA INC., Cambridge, MA (US)

(72) Inventors: Michael SOLIMAN, Concord, MA (US); Matthew P. SCARAMOZZA, West Roxbury, MA (US); Million AREFAYENE, Wayland, MA (US); Gab-Jin PARK, Belmont, MA (US); Eris BAME, Dedham, MA (US); Michael Joseph PALTE, Melrose, MA (US)

(21) Appl. No.: 19/480,964

(22) PCT Filed: May 3, 2024
(86) PCT No.: PCT/US2024/027812

§ 371 Date: Nov. 3, 2025

Related U.S. Application Data
(60) Provisional application No. 63/464,288, filed on May 5, 2023.
(60) Provisional application No. 63/541,965, filed on Oct. 2, 2023.

(51) Int. Cl. A61K 31/55; A61K 9/00

(52) U.S. Cl. CPC A61K 31/55; A61K 9/0056

(57) ABSTRACT

The present disclosure provides methods of treating Multiple Sclerosis (MS) using Compound (1) as represented by the structure below or a pharmaceutically acceptable salt thereof.

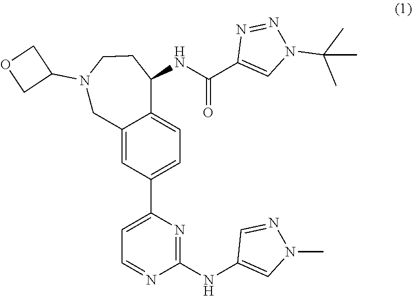 [SMILES: Cn1cc(Nc2nccc(-c3ccc4c(c3)CN(C3COC3)CC[C@H]4NC(=O)c3cn(C(C)(C)C)nn3)n2)cn1]

### RELATED APPLICATIONS

This application claims the benefit of the filing date, under 35 U.S.C. § 119(e), of U.S. Provisional Application No. 63/464,288, filed on May 5, 2023 and U.S. Provisional Application No. 63/541,965, filed on Oct. 2, 2023, the entire contents of each of above-referenced applications are incorporated herein by reference.

### BACKGROUND

Multiple sclerosis (MS) is a chronic, autoimmune, demyelinating disorder of the central nervous system (CNS) that is characterized by inflammation, demyelination, and axonal injury as well as oligodendrocyte and neuronal loss. It is the most common demyelinating disorder of the CNS, affecting approximately 2.5 million people worldwide. Relapsing multiple sclerosis (RMS) includes patients with clinically isolated syndrome (CIS), relapsing-remitting multiple sclerosis (RRMS), and active secondary progressive multiple sclerosis (SPMS). In the relapsing-remitting phase of the disease, patients experience episodes of neurological dysfunction (relapses) separated by periods of relative stability. Other types of MS include primary progressive multiple sclerosis (PPMS) and non-relapsing SPMS. Patients with radiologically isolated syndrome (RIS) may go on to develop MS.

While there has been substantial progress in MS care over the last 25 years with the approval of a number of medicinal products, many patients with MS continue to experience permanent disability progression, and many receive therapies with limited tolerability, burdensome monitoring, or risk of severe adverse events (SAEs) or even life-threatening side effects. There remains an important unmet need for tolerable MS therapies that are highly efficacious and include new options for treating the inflammatory component of MS seen across the spectrum of RMS through progressive multiple sclerosis (PMS) types, as well as effectively treating disability progression independent of relapse activity.

### SUMMARY OF THE INVENTION

The present disclosure provides methods of treating multiple sclerosis in a subject in need thereof comprising administering to the subject an effective amount of Compound 1 or a pharmaceutically acceptable salt thereof. The disclosure provides safe and effective dosing regimens for Compound 1. More specifically, the disclosure provides a total oral daily dose of 200 mg to 1000 mg Compound 1 or a pharmaceutically acceptable salt thereof in an amount equivalent to 200 mg to 1000 mg of Compound 1 for use in the methods of the present disclosure.

In one aspect, the present disclosure provides a method of treating a subject with MS, comprising administering to the subject in need thereof orally an effective amount of Compound 1 or a pharmaceutically acceptable salt thereof.

Compound 1 represented by the structure below:

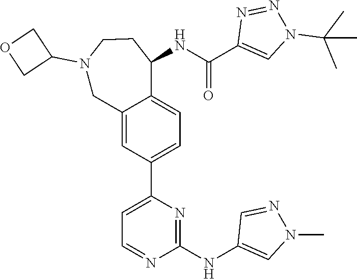 [SMILES: Cn1cc(Nc2nccc(-c3ccc4c(c3)CN(C3COC3)CC[C@H]4NC(=O)c3cn(C(C)(C)C)nn3)n2)cn1]

or a pharmaceutically acceptable salt thereof, is used in the methods of the present disclosure.

In another aspect, 200 mg to 1000 mg of Compound 1 or a pharmaceutically acceptable salt thereof in an amount equivalent to 200 mg to 1000 mg of Compound 1 is provided for oral and daily use in treating a subject with MS.

### BRIEF DESCRIPTION OF DRAWINGS

FIG. 1. shows mean plasma concentration-time profiles for BIIB091 when administered as IR table with different regimens.

FIG. 2 shows pre-dose plasma BIIB091 concentrations following administration of multiple oral doses of 250 mg IR tablets BID with MF meals.

FIG. 3 shows CD69% inhibition on total B cells (CD19+) with a 250 mg BIIB091 IR tablet BID×7 days (data up to Day 9).

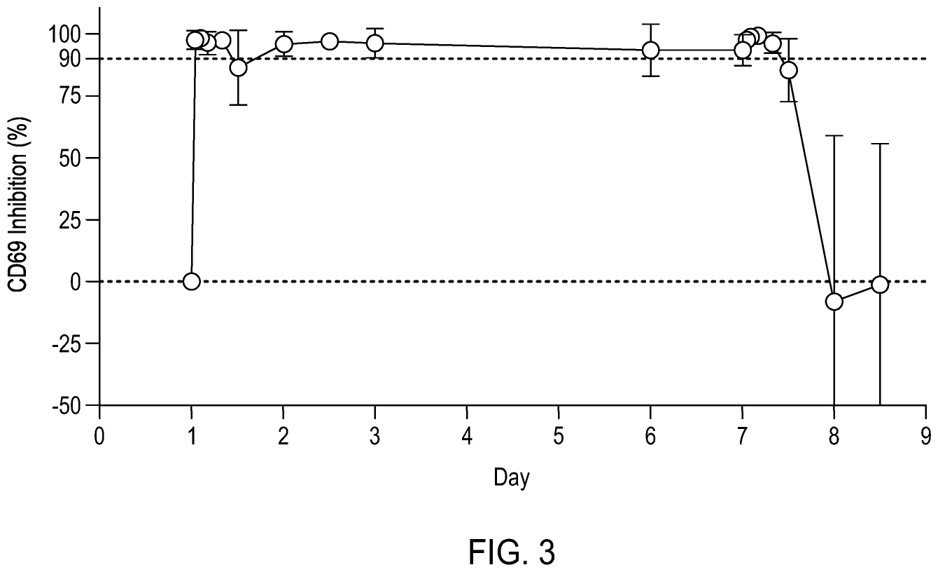

### DETAILED DESCRIPTION OF THE INVENTION

The present disclosure provides therapies for treating MS in a subject in need thereof. The methods comprise administering to the subject an effective amount of Compound 1, a BTK inhibitor. In some embodiments, Compound 1 is used as a monotherapy for treating MS.

### BTK Inhibitors

Bruton's tyrosine kinase (BTK), a member of the tyrosine kinase hepatocellular carcinoma (TEC) family of protein tyrosine kinases, is expressed in many hematopoietic cell types known to be dysregulated in MS. Additionally, pathogenic activation of B cells is considered a key driver in the maintenance of active inflammation in MS. Recent studies in patients with MS have established B cells as a clinically validated target cell type in MS. In addition to B cells, there is a body of support for the pathological role of myeloid cells (monocytes, macrophages, dendritic cells, mast cells, and granulocytes) in MS. BTK is a key signaling node immediately downstream of the B-cell receptor (BCR) in B cells and Fc receptors (FcRs) in myeloid cells. In B cells, BTK mediates B-cell activation and effector functions (such as cytokine secretion and proliferation and differentiation into memory cells and antibody-producing cells) downstream of BCR activation and is required for BCR-mediated antigen presentation to T cells. In myeloid cells, BTK inhibition blocks FcR dependent pro-inflammatory activities (including cytokine secretion by mast cells, monocytes, and macrophages; reactive oxygen species generation by neutrophils; and degranulation of basophils) triggered by binding of immune complexes to FcRs. Genetic ablation of all activating FcγRs or FcγRIII has established the pathogenic role of FcRs in myeloid cells in MS nonclinical models. The role of FcRs in disease pathogenesis is not completely understood but includes immune complex mediated endocytosis and antigen presentation to T cells as well as the regulation of myeloid cell activation and functions. Therefore, by targeting both B cells and myeloid cells, BTK inhibitors have the potential to offer additional clinical benefits as compared to therapies targeting only B cells.

In some embodiments, the BTK inhibitor is (R)-1-(tert-butyl)-N-(8-(2-((1-methyl-1H-pyrazol-4-yl)amino)pyrimidin-4-yl)-2-(oxetan-3-yl)-2,3,4,5-tetrahydro-1H-benzo[c]azepin-5-yl)-1H-1,2,3-triazole-4-carboxamide represented by the following structural formula:

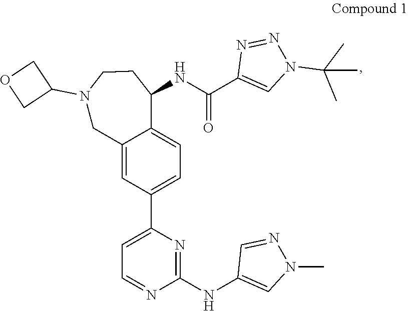 [SMILES: Cn1cc(Nc2nccc(-c3ccc4c(c3)CN(C3COC3)CC[C@H]4NC(=O)c3cn(C(C)(C)C)nn3)n2)cn1]

or a pharmaceutically acceptable salt thereof.

In some embodiments, for the methods described herein (e.g., for treating MS, relapsing form of MS, RRMS, SPMS, PPMS, CIS, RIS etc.), the BTK inhibitor is Compound 1 or a pharmaceutically acceptable salt thereof.

### Methods of Treatment

The present disclosure provides methods of treating a subject (e.g., a human patient) with MS, by administering Compound 1 to the subject.

In some embodiments, the MS is relapsing form of MS (RMS).

In some embodiments, the MS is relapsing-remitting MS (RRMS) or secondary progressive MS (SPMS).

In some embodiments, the MS is primary progressive MS (PPMS).

As used herein, a “relapsing form of MS” includes clinically isolated syndrome (CIS), relapsing-remitting disease (RRMS), and active secondary progressive disease.

In some embodiments, the methods can be used for treating MS selected from relapsing-remitting MS (RRMS), secondary progressive MS (SPMS), non-relapsing SPMS, primary progressive MS (PPMS), clinically isolated syndrome (CIS), and radiologically isolated syndrome (RIS).

CIS is a first episode of neurologic symptoms caused by inflammation and demyelination in the central nervous system. The episode, which by definition must last for at least 24 hours, is characteristic of multiple sclerosis but does not yet meet the criteria for a diagnosis of MS because people who experience a CIS may or may not go on to develop MS. When CIS is accompanied by lesions on a brain MRI (magnetic resonance imaging) that are similar to those seen in MS, the person has a high likelihood of a second episode of neurologic symptoms and diagnosis of relapsing-remitting MS. When CIS is not accompanied by MS-like lesions on a brain MRI, the person has a much lower likelihood of developing MS.

RRMS, the most common disease course of MS, is characterized by clearly defined attacks of new or increasing neurologic symptoms. These attacks—also called relapses or exacerbations—are followed by periods of partial or complete recovery (remissions). During remissions, all symptoms may disappear, or some symptoms may continue and become permanent. However, there is no apparent progression of the disease during the periods of remission. RRMS can be further characterized as either active (with relapses and/or evidence of new MRI activity over a specified period of time) or not active, as well as worsening (a confirmed increase in disability following a relapse) or not worsening.

SPMS follows an initial relapsing-remitting course. Some people who are diagnosed with RRMS will eventually transition to a secondary progressive course in which there is a progressive worsening of neurologic function (accumulation of disability) over time. SPMS can be further characterized as either active (with relapses and/or evidence of new MRI activity during a specified period of time) or not active, as well as with progression (evidence of disability accumulation over time, with or without relapses or new MRI activity) or without progression.

PPMS is characterized by worsening neurologic function (accumulation of disability) from the onset of symptoms, without early relapses or remissions. PPMS can be further characterized as either active (with an occasional relapse and/or evidence of new MRI activity over a specified period of time) or not active, as well as with progression (evidence of disability accumulation over time, with or without relapse or new MRI activity) or without progression.

Patients diagnosed with RIS do not present any overt symptoms of MS, but exhibit brain abnormality (e.g., observed by magnetic resonance imaging (MRI)) that are similar to what is seen in patients with MS. Diagnosis of RIS often occurs during a brain scan due to unrelated conditions, such as headache, migraines, head injury, stroke etc. Although there is a strong association between RIS and MS (RIS often indicates the earliest detectable preclinical phase of the disease), patients with RIS may not go on to develop MS.

As used herein, the term “pharmaceutically-acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically acceptable salts are well known in the art. For example, S. M. Berge et al. describes pharmacologically acceptable salts in J. Pharm. Sci., 1977, 66, 1-19.

As used herein, the terms “subject” and “patient” may be used interchangeably, and mean a mammal in need of treatment, e.g., a human, companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

An “effective amount” of Compound 1 is an amount sufficient to provide a therapeutic benefit in the treatment of disease or disorder described herein or to delay or minimize one or more symptoms associated with the disorder or disease, is an amount sufficient to provide a therapeutic benefit in the treatment of a disorder or disease described herein or to delay or minimize one or more symptoms associated with the disorder or disease when combined with Compound 1. The term “therapeutically effective amount” and “effective amount” are used interchangeably. The term “effective amount” can encompass an amount that improves overall therapy, reduces or avoids symptoms, signs, or causes of the condition, and/or enhances the therapeutic efficacy of another therapeutic agent. In certain embodiments, an effective amount is an amount sufficient for eliciting therapeutic effects in the treatment of MS described herein.

The present disclosure provides a method of treating a human subject with multiple sclerosis (MS), comprising administering to the subject orally a total daily dose of 200 mg to 1000 mg of Compound 1:

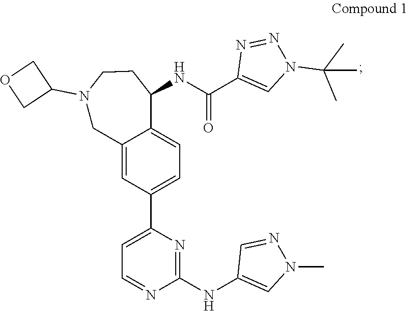 [SMILES: Cn1cc(Nc2nccc(-c3ccc4c(c3)CN(C3COC3)CC[C@H]4NC(=O)c3cn(C(C)(C)C)nn3)n2)cn1]

or a pharmaceutically acceptable salt thereof in an amount equivalent to a total daily dose of 200 mg to 1000 mg of Compound 1.

Compound 1 disclosed herein can be used in the form of a free base or as a salt. Representative salts include the hydrobromide, hydrochloride, sulfate, bisulfate, phosphate, nitrate, acetate, valerate, oleate, palmitate, stearate, laurate, benzoate, lactate, tosylate, citrate, maleate, fumarate, succinate, tartrate, naphthylate, mesylate, glucoheptonate, lactobionate, and laurylsulphonate salts and the like. (See, e.g., Berge et al. (1977) “Pharmaceutical Salts”, J. Pharm. Sci. 66:1-19).

In some embodiments, in the methods of treatment disclosed herein, Compound 1 is administered as the free base. In some embodiments, in the methods of treatment disclosed herein, a pharmaceutically acceptable salt of Compound 1 is administered.

When the stereochemical configuration at a chiral center in Compound 1 is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

### Pharmaceutical Compositions

Pharmaceutical compositions of the disclosure (also referred to herein as the “disclosed pharmaceutical compositions”) comprise one or more pharmaceutically acceptable carrier(s) or diluent(s) and Compound 1, or a pharmaceutically acceptable salt thereof.

“Pharmaceutically acceptable carrier” and “pharmaceutically acceptable diluent” refer to a substance that aids the formulation and/or administration of an active agent to and/or absorption by a subject and can be included in the pharmaceutical compositions of the disclosure without causing a significant adverse toxicological effect on the subject. Non-limiting examples of pharmaceutically acceptable carriers and/or diluents include NaCl, lactated Ringer's, normal sucrose, normal glucose, binders, fillers, disintegrants, lubricants, coatings, sweeteners, flavors, alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds or pharmaceutically acceptable salts thereof.

The pharmaceutical compositions of the disclosure optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Administration and Dosage Forms

The precise amount of compound administered to provide an “effective amount” to the subject will depend on the type, and severity of the MS, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-MS agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of Compound 1 being used by following, for example, dosages reported in the literature and recommended in the Physician's Desk Reference (57th Ed., 2003).

In some embodiments, the subject in need thereof is administered a total daily dose of 300 mg to 900 mg of Compound 1.

In some embodiments, the subject in need thereof is administered a total daily dose of 400 mg to 800 mg of Compound 1.

In some embodiments, the subject in need thereof is administered a total daily dose of 500 mg to 700 mg of Compound 1.

In some embodiments, the subject in need thereof is administered a total daily dose of 200 mg to 400 mg, 300 mg to 500 mg, 400 mg to 600 mg, 500 mg to 700 mg, 600 mg to 800 mg, 700 mg to 900 mg, 800 mg to 1000 mg, 450 mg to 550 mg or 650 mg to 750 mg of Compound 1.

In some embodiments, the subject in need thereof is administered a total daily dose of 200 mg of Compound 1.

In some embodiments, the subject in need thereof is administered a total daily dose of 300 mg of Compound 1.

In some embodiments, the subject in need thereof is administered a total daily dose of 400 mg of Compound 1.

In some embodiments, the subject in need thereof is administered a total daily dose of 500 mg of Compound 1.

In some embodiments, the subject in need thereof is administered a total daily dose of 600 mg of Compound 1.

In some embodiments, the subject in need thereof is administered a total daily dose of 700 mg of Compound 1.

In some embodiments, the subject in need thereof is administered a total daily dose of 800 mg of Compound 1.

In some embodiments, the subject in need thereof is administered a total daily dose of 900 mg of Compound 1.

In some embodiments, the subject in need thereof is administered a total daily dose of 1000 mg of Compound 1.

In some embodiments, the subject in need thereof is administered a total daily dose of 200 mg, 225 mg, 250 mg, 275 mg, 300 mg, 325 mg, 350 mg, 375 mg, 400 mg, 425 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 825 mg, 850 mg, 900 mg, 925 mg, 950 mg, 975 mg, or 1000 mg of Compound 1.

In some embodiments, the subject in need thereof is administered Compound 1 once daily (QD) orally with the amount identified above.

In some embodiments, the subject in need thereof is administered Compound 1 twice daily (BID) orally with the amount identified above.

In some embodiments, the subject in need thereof is administered an amount of 100 mg to 500 mg of Compound 1 twice daily (BID). The total daily dose is 200 mg to 1000 mg of Compound 1.

In some embodiments, the subject in need thereof is administered an amount of 150 mg to 450 mg of Compound 1 twice daily (BID). The total daily dose is 300 mg to 900 mg of Compound 1.

In some embodiments, the subject in need thereof is administered an amount of 200 mg to 400 mg of Compound 1 twice daily (BID). The total daily dose is 400 mg to 800 mg of Compound 1.

In some embodiments, the subject in need thereof is administered an amount of 250 mg to 350 mg of Compound 1 twice daily (BID). The total daily dose is 500 mg to 700 mg of Compound 1.

In some embodiments, the subject in need thereof is administered an amount of 100 mg to 200 mg, 150 mg to 250 mg, 200 mg to 300 mg, 250 mg to 350 mg, 300 mg to 400 mg, 350 mg to 450 mg, 400 mg to 500 mg, 225 mg to 275 mg, or 325 mg to 375 mg of Compound 1 twice daily (BID).

In some embodiments, the subject in need thereof is administered an amount of 200 mg of Compound 1 twice daily (BID).

In some embodiments, the subject in need thereof is administered an amount of 250 mg of Compound 1 twice daily (BID).

In some embodiments, the subject in need thereof is administered an amount of 275 mg of Compound 1 twice daily (BID).

In some embodiments, the subject in need thereof is administered an amount of 300 mg of Compound 1 twice daily (BID).

In some embodiments, the subject in need thereof is administered an amount of 325 mg of Compound 1 twice daily (BID).

In some embodiments, the subject in need thereof is administered an amount of 350 mg of Compound 1 twice daily (BID).

In some embodiments, the subject in need thereof is administered an amount of 375 mg of Compound 1 twice daily (BID).

In some embodiments, the subject in need thereof is administered an amount of 400 mg of Compound 1 twice daily (BID).

In some embodiments, the subject in need thereof is administered an amount of 450 mg of Compound 1 twice daily (BID).

In some embodiments, the subject in need thereof is administered an amount of 500 mg of Compound 1 twice daily (BID).

In some embodiments, the subject in need thereof is administered an amount of 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg or 500 mg of Compound 1 twice daily (BID).

In some embodiments, the subject in need thereof is administered Compound 1 with food (e.g., a regular meal, a moderate fat meal, a high fat meal, etc.). In some embodiments, the subject in need thereof is administered Compound 1 with a moderate fat meal. In some embodiments, the moderate fat meal has a total calorie of 500-800 Kcal. In some embodiments, the moderate fat meal has between 14 and 55 g of fat, and/or between 25% and 50% by weight of the meal is fat, and/or total calorie for the fat content in the meal is between 125 and 500 Kcal. In some embodiments, the subject in need thereof is administered Compound 1 after food (e.g., a regular meal, a moderate fat meal, etc.). In one embodiment, the subject in need thereof is administered Compound 1 shortly after food (e.g., a regular meal, a moderate fat meal, etc.), for example, less than 4 hours, less than 2 hours, less than 1 hours or less than 30 minutes after food (e.g., a regular meal, a moderate fat meal, etc.). In some embodiments, the subject in need thereof is administered Compound 1 between 30 and 60 minutes after food (e.g., a regular meal, a moderate fat meal, etc.).

In some embodiments, the subject in need thereof is administered Compound 1 in a fasted state.

In some embodiments, an immediate release (IR) formulation of Compound 1 in tablets is used in the methods of the present disclosure. In some embodiments, the tablets contain 50 mg, 150 mg or 250 mg of Compound 1. In some embodiments, the immediate release formulation of Compound 1 comprising one or more excipients selected from silicified microcrystalline cellulose, sodium starch glycolate, sodium stearyl fumarate, and colloidal silicon dioxide. In some embodiments, the immediate release tablet comprises a film coating comprising titanium dioxide, hypromellose, macrogol, iron oxide yellow, and ferroferric oxide.

In some embodiments, for the methods described herein, the subject is administered Compound 1 in the absence of a cytochrome P450 3A4 (CYP3A4) inhibitor.

Administering “in the absence of a CYP3A4 inhibitor” means that the subject was never taking a CYP3A4 inhibitor or stopped taking a CYP3A4 inhibitor before treatment with Compound 1 or a pharmaceutically acceptable salt thereof was initiated or stopped taking a CYP3A4 inhibitor at the time treatment with Compound 1 or a pharmaceutically acceptable salt thereof was initiated. When the subject is taking a CYP3A4 inhibitor, it is desirable that administration of the CYP3A4 inhibitor be terminated at 1, 2, 3, 4, 5 or 6 days before initiation of treatment with Compound 1 or a pharmaceutically acceptable salt thereof, or at least 1, 2, 3, 4, 5, 6, 7, 8 or more weeks before initiation of treatment with Compound 1 or a pharmaceutically acceptable salt thereof, or at least 1, 2 or 3 months before initiation of treatment with Compound 1 or a pharmaceutically acceptable salt thereof.

A CYP3A4 inhibitor is a substance that decreases the activity of CYP3A4. In some embodiments, the CYP3A4 inhibitor is a strong inhibitor of CYP3A4, such as clarithromycin, indinavir, nefazodone, saquinavir, suboxone, telithromycin, erythromycin, diltiazem, itraconazole, ketoconazole, ritonavir, and goldenseal. Intermediate strength inhibitors include aprepitant, erythromycin, fluconazole, grapefruit, verapamil, and diltiazem. In some embodiments, the CYP3A4 inhibitor is a weak inhibitor of CYP3A4, such as cimetidine. Other exemplary CYP3A4 inhibitors include, but are not limited to amiodarone, boceprevir, chloramphenicol, ciprofloxacin, delaviridine, diethyl-dithiocarbamate, fluvoxamine, gestodene, imatinib, mibefradil, mifepristone, norfloxacin, norfluoxetine, starfruit, telaprevir, and voriconazole. In some embodiments, the CYP3A4 inhibitor is itraconazole.

In some embodiments, for the methods described herein, the subject is administered Compound 1 in the absence of a proton pump inhibitor (PPI). In some embodiments, for the methods described herein, the subject is administered Compound 1 in the absence of a proton pump inhibitor (PPI) when the subject is in fasted state. In some embodiments, for the methods described herein, the subject is administered Compound 1 and a proton pump inhibitor (PPI) with or shortly after food (e.g., a regular meal, a moderate fat meal, a high fat meal, etc.).

Administering “in the absence of a PPI” means that the subject was never taking a PPI or stopped taking a PPI before treatment with Compound 1 or a pharmaceutically acceptable salt thereof was initiated or stopped taking a PPI at the time treatment with Compound 1 or a pharmaceutically acceptable salt thereof was initiated. When the subject is taking PPI, it is desirable that administration of the PPI be terminated at 1, 2, 3, 4, 5 or 6 days before initiation of treatment with Compound 1 or a pharmaceutically acceptable salt thereof, or at least 1, 2, 3, 4, 5, 6, 7, 8 or more weeks before initiation of treatment with Compound 1 or a pharmaceutically acceptable salt thereof, or at least 1, 2 or 3 months before initiation of treatment with Compound 1 or a pharmaceutically acceptable salt thereof.

A PPI is a class of drugs that cause a profound and prolonged reduction of stomach acid production by irreversibly inhibiting the stomach's H+/K+ ATPase proton pump. Exemplary PPIs include, but are not limited to omeprazole, lansoprazole, dexlansoprazole, esomeprazole, pantoprazole, rabeprazole, and ilaprazole. In some embodiments, the PPI is rabeprazole.

### EXEMPLIFICATION

### Example 1. Phase 2 Clinical Study

### Study Design

This is a multicenter, randomized, blinded, active controlled Phase 2 study to evaluate the safety and efficacy of Compound 1 in participants with RMS. The primary objective of the study is to investigate the safety and tolerability of BIIB091 monotherapy in participants with RMS with the primary endpoint for the study being incidence of adverse events (AEs) and SAEs. The secondary object of the study is to evaluate the effects of BIIB091 monotherapy on the MRI measures of active CNS inflammation.

The study will include a 4-week screening period, a 16-week double-blind active-controlled treatment period, a 32-week blinded active controlled treatment period, and a 2-week post-treatment safety follow-up period. Participants with absolute lymphocyte count (ALC)<lower limit of normal (LLN) at the 2 week safety Follow-Up Visit will return in 2 weeks for retesting and confirmation, and follow-up will be extended for those participants at intervals of every 8 weeks to monitor their lymphocyte counts until their ALC>LLN, or for a period up to 6 months, or until they commence another disease-modifying therapy, whichever occurs first.

Participants with active RMS will be randomized to 3 treatment groups: high-dose Compound 1 monotherapy (350 mg BID), low-dose Compound 1 monotherapy (250 mg BID), and DRF monotherapy at standard dose (462 mg BID). An analysis of primary, secondary, and selected exploratory endpoints of Compound 1 monotherapy study will be performed. An independent data monitoring committee (IDMC) will review the safety and lab data from the analysis of Compound 1 monotherapy. In the event of unfavorable safety findings, the IDMC could recommend pausing or stopping a cohort or the study or a modification to the study design. Details regarding the IDMC review of data will be provided in the IDMC charter.

Final analysis of the 48-week data from Compound 1 monotherapy will allow for the assessment of longer-term safety and efficacy of Compound 1 monotherapy. For maintenance of blinding, members of the study management team who are not involved in the Week 16 data review and the study sites will remain blinded for the entire duration of the study. Details on how the study blind is maintained will be provided in a separate unblinding plan.

Participants will visit the study site for safety, MRI, and clinical efficacy assessments at Day 28 to Day 1 (MRI assessments should be completed at least 7 calendar days and no more than 14 calendar days prior to Baseline [Day 1]), and Weeks 4, 8, 12, 16, 24, and 48. Additional specific safety assessments will be performed at Weeks 1, 2, 6, 36, and 50.

During the study, if an MS relapse is suspected, the participant should return to the study site for an unscheduled visit and be evaluated within 72 hours of the onset of the event to determine if a relapse has occurred. Treatment of an acute relapse event may proceed at the discretion of the treating neurologist only after the examining neurologist has completed their examination. The treatment for relapse in this study is intravenous methylprednisolone (IVMP)≤1000 mg/day for up to a maximum of 5 days with or without an oral prednisone taper (up to 15 days). Any changes to this treatment should first be discussed with the Study Medical Director or designee. If the start of a treatment for a relapse with high dose corticosteroids falls within 7 days of the next scheduled visit, every attempt should be made to obtain the MRI before administration of the first dose of high-dose corticosteroids. If outside the visit window, visit should be recorded as unscheduled. The MRI at an unscheduled visit prior to steroid treatment must be ≥21 days after the prior MRI. However, if an MRI at an unscheduled visit is <21 days after a prior MRI, then the use of Gd should be strongly avoided, unless determined by the Investigator to be clinically indicated. In this scenario, the next regularly scheduled MRI should also be obtained.

Study Location: Approximately 80 sites globally are planned.

Study Population: This study will be conducted in participants who meet the following criteria:
Aged 18 through 55 years old, inclusive, at the time of informed consent
Time since MS symptom onset <20 years
Diagnosed with MS per the 2010 or 2017 McDonald's criteria [Polman 2011; Thompson 2018]
Must have Expanded Disability Status Scale (EDSS) score of 0 through 5.0 at Screening
Must have at least 1 of the following occurring prior to Baseline (Day 1):
   ≥2 clinical relapses in the last 24 months (but not within 30 days prior to Baseline [Day 1]) with at least 1 relapse during the last 12 months prior to randomization
   ≥1 clinical relapse within the past 24 months (but not within 30 days prior to Baseline [Day 1]) and ≥1 new brain MRI lesion (Gd positive and/or new or enlarging T2 hyperintense lesion) within the past 12 months prior to randomization. The baseline MRI could be used to satisfy this criterion (local MRI readings are allowed). For new or enlarging T2 hyperintense lesions, the reference scan cannot be >12 months prior to randomization
   ≥1 GdE lesion on brain MRI within 6 months prior to randomization

Number of Planned Participants: Approximately 275 participants will be randomized.

Treatment Groups: Participants will be randomized into treatment groups. The randomization will be stratified by intensive PK cohort (Yes/No) and region (Eastern Europe vs. Other). For stratification, Eastern Europe will include participants from countries such as Poland and the Czech Republic.

Compound 1 monotherapy:
High dose (350 mg) BID (N=50)
Low dose (250 mg) BID (N=50)

DRF monotherapy:
Standard dose (462 mg) BID (N=50)

Sample Size Determination: The planned sample size is 275 participants. Accounting for a 12% dropout rate, 275 participants are planned to be enrolled in the study. Of these 150 will be randomized in a separate Compound 1 combination therapy study.

A sample size of 50 participants (44 evaluable) per treatment group will allow for an 80% or greater probability of observing at least 1 occurrence of an AE with an event rate of 3.6%. Intensive PK will be collected in up to 25 participants (approximately 10 participants in each BIIB091 group and approximately 5 participants in the DRF group.

Visit Schedule: Participants will have up to 12 scheduled visits during the study. Study assessments conducted at each visit are listed in the Schedule of Activities.

Visit days are calculated with respect to Day 1 (the date of first dose).

Duration of Study Participation: The study duration for participants will be approximately 54 weeks and includes the following:
4-week screening period
16-week double-blind, active-controlled treatment period
32-week blinded, active-controlled treatment period
2-week post-treatment safety follow-up period

### Example 2. Pharmacokinetic Study for Optimization of Formulation and Treatment Regimen

In a single center, open-label study, several formulations and treatment regimens were explored to reduce pharmacokinetic (PK) variability and achieve steady state C_{trough}>221 ng/mL while limiting the drug exposure (Cₘₐₓ). The study included 3 parts.

### Part 1

IR tablet (50 mg) was tested in 19 healthy subjects at different dose levels (150, 250 and 350 mg) and in the fed (moderate fat and high fat) and fasted state and as a divided daily dose (12 h apart). Subject were treated with BIIB091 up to 4 periods. To facilitate comparisons between different regimens, subjects were randomized into 2 groups (Group 1 and Group 2) pre-dose on Day 1 in Periods 1, 3 and 4. Period 2 was utilized as a reference period, where all subjects were dosed with the BIIB091 IR DiC (drug in capsule).

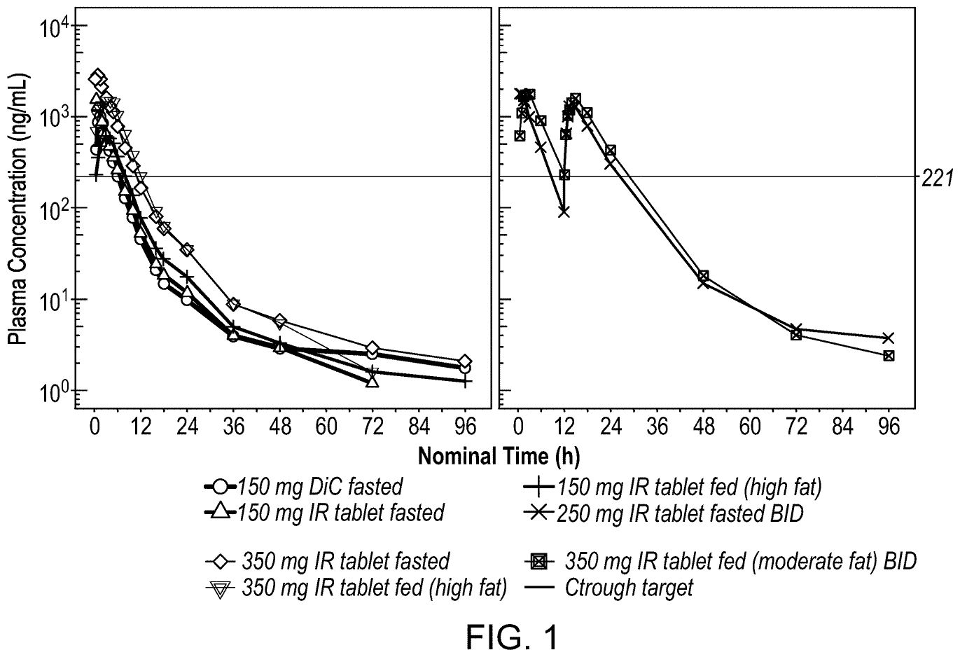

IR tablet (150 mg) had higher exposure (129% Cmax and 138% AUCinf) and lower variability (CV %) ranging from 17.1% to 30.8% for Cmax and AUCinf, compared with 43.4% to 78.1% for the DiC 150 mg reference. Two doses of 250 mg (fasted) and 350 mg (with MF) IR tablets administered 12 h apart gave geometric mean C₂₄ (CV %) of 282 (44.7%) and 411 (31.1%) ng/mL, respectively, both above the target C_{trough}. Dosing with HF food delayed T and reduced C but had similar overall exposure. Dosing with a moderate fat (MF) meal, resulted in less of a delay in Tₘₐₓ compared to high fat (HF), but had similar C and AUC All regimens of BIIB091 tested were considered to be generally safe and well tolerated. FIG. 1 shows plasma concentration of BIIB091 over time.

### Part 2

A new cohort of 12 healthy male and female (non pregnant and non-lactating) subjects were enrolled and dosed. Subjects were randomized into two groups of 6 subjects, to receive the regimen sequence MNO across 3 study periods (Group 1) or regimen sequence MNP across 3 study periods (Group 2). Both groups were dosed in the fed state in Periods 1 and 2, i.e. BIIB091 alone in the fed state in Period 1, and BIIB091 with the CYP3A4 inhibitor itraconazole in the fed state in Period 2. For the PPI assessment in Period 3, subjects were dosed with BIIB091 co-administered with rabeprazole in the fasted or fed state in order to assess PK under each of these conditions.

TABLE 1
Study Regimens for Part 2

| | | | Dose (Dose | |
|---|---|---|---|---|
| Period | Regimen | IMP/NIMP | Units) | Route of Administration |
| 1 | M | BIIB091 IR Tablet | 200 mg | Oral, Fed (moderate-fat |
| | (N = 12) | | (4 × 50 mg) | breakfast^{a}) |
| | [Group 1 | | | |
| | and | | | |
| | Group 2] | | | |
| 2 | N | Itraconazole 100 mg Capsules, hard, | 200 mg | Oral, Fed (high-fat |
| | (N = 12) | BID for 1 day (Day −4), then QD for | (2 × 100 mg) | breakfast^{b} on Days −4 |
| | [Group 1 | 3 days (Day −3, −2, and −1) | | to −2; standard evening |
| | and | | | meal on Day −4; and |
| | Group 2] | | | moderate-fat breakfast^{a} |
| | | | | on Day −1) |
| | | +Itraconazole 100 mg Capsules, hard, | 200 mg | Oral, Fed (moderate-fat |
| | | and BIIB091 IR Tablet on the 5^{th} day | (2 × 100 mg) | breakfast^{a}) |
| | | (Day 1) | and 200 mg | |
| | | | (4 × 50 mg) | |
| | | +Itraconazole 100 mg Capsules, hard, on | 200 mg | Oral, Fed (standard |
| | | the 6^{th} day (Day 2) | (2 × 100 mg) | breakfast^{c}) |
| 3 | O | Rabeprazole 20 mg Gastro-resistant | 20 mg | Oral, Fed (standard |
| | (N = 6) | Tablet, BID for 3 days (Day −3, −2, | (1 × 20 mg) | breakfast^{c}, and standard |
| | [Group 1 | and −1) | | evening meal) |
| | only] | +Rabeprazole 20 mg Gastro-resistant | 20 mg | Oral, Fasted^{d} |
| | | Tablet and BIIB091 IR Tablet on the 4^{th} | (1 × 20 mg) | |
| | | day (Day 1) | and 200 mg | |
| | | | (4 × 50 mg) | |
| | P | Rabeprazole 20 mg Gastro-resistant | 20 mg | Oral, Fed (standard |
| | (N = 6) | Tablet, BID for 3 days (Day −3, −2, | (1 × 20 mg) | breakfast^{c}, and standard |
| | [Group 2 | and −1) | | evening meal) |
| | only] | +Rabeprazole 20 mg Gastro-resistant | 20 mg | Oral, Fed (moderate-fat |
| | | Tablet and BIIB091 IR Tablet on the 4^{th} | (1 × 20 mg) | breakfast^{a}) |
| | | day (Day 1) | and 200 mg | |
| | | | (4 × 50 mg) | |

NIMP: non-investigational medicinal product, QD: once daily.
^{a}Moderate-fat breakfast comprised of 19 g protein, 87 g carbohydrates and 27 g (36%) fat
^{b}High-fat breakfast comprised of 35 g protein, 65 g carbohydrates and 61 g (57%) fat
^{c}Standard breakfast comprised of 16 g protein, 87 g carbohydrates and 17 g (27%) fat
^{d}Fasted subjects had an overnight fast for a minimum of 10 hours before the dose in each study period, and received a standard breakfast 2 hours post-dose.

The data showed that BIIB091 is a moderate CYP3A4 substrate. CYP3A4 inhibitor co-administration increased BIIB091 exposure. PPI co-administration decreased BIIB091 bioavailability in fasted state, while taking PPI and BIIB091 together with food increased BIIB091 bioavailability compared with fasted condition.

### Part 3

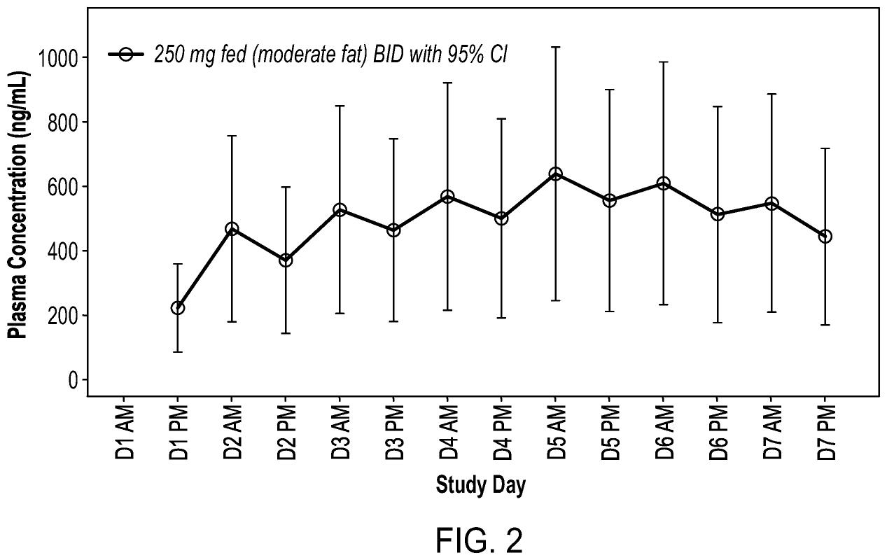

250 mg BIIB091 was dosed in a new cohort of 10 healthy subjects twice a day (BID) 12 h apart with a moderate fat meal for 7 days. Accumulation appeared moderate following BID dosing of BIIB091 for 7 days, with accumulation ratios of 1.49 and 1.75 for Cₘₐₓ and AUCₜₐᵤ respectively. Geomean Cₜₐᵤ (CV %) was 407 (48.3%) ng/mL which exceeded the target of >221 ng/mL, with 9/10 subjects achieving the target. On Day 1 and 7, complete inhibition (mean >90% from a Day 1 pre-dose baseline) of CD69 upregulation upon ex vivo stimulated B cells was observed from 1 h post-administration to 8 h post-dose (ranging from 96.5 to 99.2%), See FIG. 2. CD69 mean inhibition decreased slightly at 12 h post-dose on day 1 and 7 to 86.8% and 85.4% respectively. At 24 h post final dose, CD69 inhibition returned to baseline. IR Tablet 250 mg administered BID for 7 days in the fed state was considered to be safe and well tolerated.

What is claimed is:

1. A method of treating a human subject with multiple sclerosis (MS), comprising administering to the subject orally a total daily dose of 200 mg to 1000 mg of Compound 1: 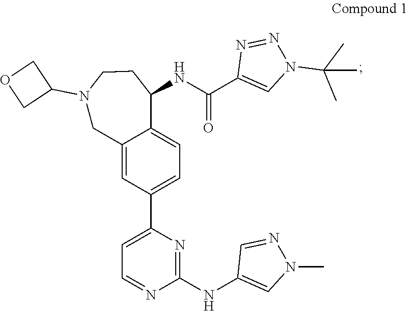 [SMILES: Cn1cc(Nc2nccc(-c3ccc4c(c3)CN(C3COC3)CC[C@H]4NC(=O)c3cn(C(C)(C)C)nn3)n2)cn1]
or a pharmaceutically acceptable salt thereof in an amount equivalent to a total daily dose of 200 mg to 1000 mg of Compound 1.

2. The method of claim 1, wherein the MS is relapsing form of MS.

3. The method of claim 1, wherein MS is relapsing-remitting MS (RRMS) or secondary progressive MS (SPMS).

4. The method of claim 1, wherein the MS is primary progressive MS (PPMS).

5. The method of any one of claims 1-4, wherein the subject is administered a total daily dose of 300 mg to 900 mg of Compound 1.

6. The method of any one of claims 1-4, wherein the subject is administered a total daily dose of 400 mg to 800 mg of Compound 1.

7. The method of any one of claims 1-4, wherein the subject is administered a total daily dose of 500 mg to 700 mg of Compound 1.

8. The method of any one of claims 1-4, comprising administering to the subject a total daily dose of 200 mg of Compound 1.

9. The method of any one of claims 1-4, comprising administering to the subject a total daily dose of 300 mg of the Compound 1.

10. The method of any one of claims 1-4, comprising administering to the subject a total daily dose of 400 mg of the Compound 1.

11. The method of any one of claims 1-4, comprising administering to the subject a total daily dose of 500 mg of the Compound 1.

12. The method of any one of claims 1-4, comprising administering to the subject a total daily dose of 600 mg of the Compound 1.

13. The method of any one of claims 1-4, comprising administering to the subject a total daily dose of 700 mg of the Compound 1.

14. The method of any one of claims 1-4, comprising administering to the subject a total daily dose of 800 mg of the Compound 1.

15. The method of any one of claims 1-4, comprising administering to the subject a total daily dose of 900 mg of the Compound 1.

16. The method of any one of claims 1-4, comprising administering to the subject a total daily dose of 1000 mg of the Compound 1.

17. The method of any one of claims 1-16, wherein Compound 1 is administered to the subject once daily.

18. The method of any one of claims 1-16, wherein Compound 1 is administered to the subject twice daily.

19. The method of claim 18, wherein 100 mg to 500 mg of Compound 1 is administered to the subject twice daily.

20. The method of claim 18, wherein 150 mg to 450 mg of Compound 1 is administered to the subject twice daily.

21. The method of claim 18, wherein 200 mg to 400 mg of Compound 1 is administered to the subject twice daily.

22. The method of claim 18, wherein 250 mg to 350 mg of Compound 1 is administered to the subject twice daily.

23. The method of claim 18, wherein 200 mg of Compound 1 is administered to the subject twice daily.

24. The method of claim 18, wherein 250 mg of Compound 1 is administered to the subject twice daily.

25. The method of claim 18, wherein 275 mg of Compound 1 is administered to the subject twice daily.

26. The method of claim 18, wherein 300 mg of Compound 1 is administered to the subject twice daily.

27. The method of claim 18, wherein 325 mg of Compound 1 is administered to the subject twice daily.

28. The method of claim 18, wherein 350 mg of Compound 1 is administered to the subject twice daily.

29. The method of claim 18, wherein 375 mg of Compound 1 is administered to the subject twice daily.

30. The method of claim 18, wherein 400 mg of Compound 1 is administered to the subject twice daily.

31. The method of claim 18, wherein 450 mg of Compound 1 is administered to the subject twice daily.

32. The method of claim 18, wherein 500 mg of Compound 1 is administered to the subject twice daily.

33. The method of any one of claims 1-32, wherein Compound 1 is administered as monotherapy.

34. The method of any one of claims 1-33, wherein Compound 1 is administered to the subject right after a regular meal.

35. The method of any one of claims 1-34, wherein compound 1 is administered to the subject in the absence of a CYP3A4 inhibitor.

36. The method of claim 35, wherein the CYP3A4 inhibitor is itraconazole.
